(19) United States
(12) Patent Application Publication
(10) Pub. No.: US 2004/0156799 A1
(43) Pub. Date: Aug. 12, 2004

(54) Cancer treatment method and compositions

(76) Inventors: Zigang Dong, Austin, MN (US); Ann M. Bode, Cannon Falls, MN (US); Wei-Ya Ma, Austin, MN (US)

Correspondence Address: Schwegman Lundberg, Woessner & Kluth, PO Box 2938, Minneapolis, MN, 55402, US

(21) Appl. No.: 10/479,899

(22) PCT Filed: Jun. 4, 2002
(86) PCT No.: PCT/US02/17705

(51) Int. Cl. A61K 7/42; A61K 31/12; A61K 31/22

(52) U.S. Cl. 424/59; 514/546; 514/678

(57) ABSTRACT

The present invention provides pharmaceutical compositions including: a combination of a compound of the formula (I) wherein R¹ is C₁-C₆ alkyl and R² is independently —H or —OH, or a pharmaceutically acceptable salt thereof; and a phorbol compound. The present invention also provides for these and other compositions and their use for the treatment of established cancers or tumors.

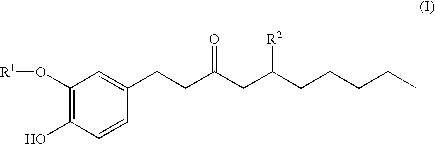

### CROSS-REFERENCE TO RELATED APPLICATION(S)

This application claims priority to U.S. Provisional Patent Application Serial No. 60/296,098, filed Jun. 5, 2001, entitled “THERAPEUTIC REGIMEN UTILIZING COMPOUNDS OF THE GINGER FAMILY TO TREAT ESTABLISHED CANCERS”.

### FIELD OF THE INVENTION

The present invention relates generally to therapeutic compositions and methods of use in treating cancer, and more specifically the present invention relates to therapeutic compositions and methods of use which are effective in arresting the growth of tumors, shrinking the size of established tumors, or clearing tumors entirely in mammalian cancers, for example, of the skin and other organ tissues.

### BACKGROUND OF THE INVENTION

Carcinoma is one of the most serious diseases threatening human and animal health and life. Predominant treatments for cancer patients are radiotherapy and chemotherapy. Both treatments have certain known toxicity or side effects on humans while suppressing cancer cell growth or killing cancer cells. Extensive investigations have been carried out in order to find effective anti-carcinogen compounds with minimum side effects and toxicity.

Sunlight, which includes ultraviolet wavelength components, is a known causal factor of certain mammalian skin cancers. Similarly, a host of chemical compounds are known causal factors, for initiation or promotion, of certain mammalian cancers of the skin and other organ tissues.

Prior studies have examined the chemo-preventive effects of ginger and focused on the effect of ginger extracts on phorbol ester, that is 12-O-tetradecanoyl-phorbol-13-acetate(TPA), or TPA-induced tumor promotion. Topical pre-application of an ethanol extract of the ginger root was shown to inhibit TPA induced epidermal ornithine decarboxylase (ODC) activity and protein expression, and TPA-induced epidermal lipo-oxygenase activity in the skin of mice. In addition, topical application of the ginger extract prior to TPA-induced tumor promotion inhibited the development of tumors in 7,12-dimethylbenz[a]anthracene (DMBA)-initiated mice. In a more recent study, 6-gingerol was used to investigate its protective effects against TPA-induced skin tumor formation in mice. The results indicated that the 6-gingerol inhibited TPA-induced skin tumor promotion in addition to inhibiting ear edema and epidermal ODC activity. There is evidence to suggest that 6-gingerol has strong antioxidant capacity which contributes to its anti-cancer effects. At least two recent studies suggested that 6-gingerol and 6-paradol suppressed proliferation of human cancer cells through the induction of apoptosis. These reports focused on the use of 6-gingerol and 6-paradol as potential chemo-preventive agents and none of the existing studies used either compound to treat existing cancers or in combination with each other or with TPA to reduce the size of an established tumor. Aside from these studies little is known about the cellular or molecular mechanisms by which gingerol or paradol may exert anti-carcinogenic effects. See for example, Y.-J. Surh, Mutation Research, 428, (1999) 305-327.

There remains a need for therapeutic compositions and methods for treating cancer, especially for compositions and methods which are effective in arresting the growth of established skin cancer tumors, shrinking the size of established tumors, or completely eliminating established skin cancer tumors, in mammals including humans. There is also a need for pharmacological tools for further study of the physiological processes associated with mammalian cancer.

### SUMMARY OF THE INVENTION

Applicants have unexpectedly discovered therapeutic compositions and treatment methods which can arrest established tumor growth, shrink the size of established tumors, or completely eliminate established tumors in mammalian skin cancer. The present invention provides in embodiments a therapeutic regimen which uses compounds of the Ginger (Zingiber officinale Roscoe, Zingiberaceae) family to treat existing or established cancers. The therapeutic compositions, in embodiments, can include one or more phenolic compounds, such as either or both 6-gingerol and 6-paradol. In embodiments, the therapeutic compositions, can include a tumor promoting compound, such as phorbol myristate acetate known as TPA, in combination with one or more of the above mentioned phenolic compounds. The resulting mixtures are synergistically highly effective and useful in methods for treating tumors, for example, treatment of established mammalian skin cancers. It was also unexpectedly discovered that any combination of the TPA tumor promoter compound, and either of the phenolic compounds, 6-gingerol and 6-paradol, when administered at any suitable dose level was highly effective in arresting and shrinking tumors but was found to be non-toxic and had no adverse side-effects in mammals, for example in mice, where there was observed no extraordinary mortality or disease symptoms associated with the treatment regimens.

Accordingly the present invention includes:

A pharmaceutical composition comprising a combination of a compound of the formula (I)

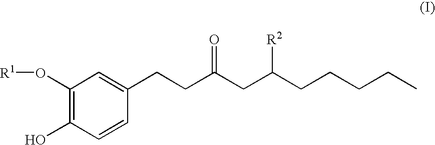

wherein R¹ is C₁-C₆ alkyl and R² is independently —H or —OH, or a pharmaceutically acceptable salt thereof; and a phorbol compound; and a pharmaceutically acceptable carrier;

A pharmaceutical composition comprising 6-gingerol and 12-O-tetradecanoyl-phorbol-13-acetate, and a pharmaceutically acceptable carrier;

A pharmaceutical composition comprising 6-paradol and 12-O-tetradecanoyl-phorbol-13-acetate, and a pharmaceutically acceptable carrier; and

A method for the treatment of cancer, such as chemically or radiation induced or promoted skin tumors, comprising:

administering to a mammal in need of such treatment a therapeutically effective amount of a combination, such as together, separately, or sequentially, of a compound or compounds of the formula (I)

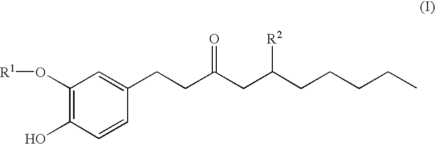

wherein R¹ is C₁-C₆ alkyl and R² is independently —H or —OH, or a pharmaceutically acceptable salt thereof; and a phorbol compound, such as phorbol diester, and the like compounds.

The invention also provides a pharmaceutical composition comprising a compound of the above formula (I), or a pharmaceutically acceptable salt thereof, and a phorbol compound, in combination with a pharmaceutically acceptable diluent or carrier.

The invention also provides for any of the above compounds or combinations thereof for use in medical therapy, for example, in treating various mammalian tumors, as well as the use of a compound of formula (I) for the manufacture of a medicament useful for the treatment of cancer, such as various tumors in a mammal, such as a human.

These and other aspects of the present invention are illustrated herein.

### Definitions

The following definition(s) are used, unless otherwise described:

Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

“Combination” in the context of administration means combined treatment where the active compounds are provided to the mammal, for example, together, separately, or sequentially, but within such a time frame that the compound combination effecilveiy acts Logetner or in concert.

“A therapeutically effective amount” means the concentration or quantity or level of the compound of the present invention that can attain a particular medical end, such as control or destruction of cancer cells, virally-infected cells, or viruses without producing unacceptable toxic symptoms. The term “safe and effective amount” refers to the quantity of a component which is sufficient to yield a desired therapeutic response without undue adverse side effects, such as toxicity, irritation, or allergic response, commensurate with a reasonable benefit/risk ratio when used in the manner of this invention. The specific “safe and effective amount” will vary with such factors as the particular condition being treated, the physical condition of the patient, the type of mammal being treated, the duration of the treatment, the nature of concurrent therapy, if any, and the specific formulations employed and the compound(s) selected or their salts.

“In need of such treatment” means a mammal having cancer or cancerous condition, such as a skin tumor.

“Free of toxic effects” means, for example, in treated mammals, such as mice, there was observed no mortality or disease symptoms associated with the treatment regimens. Most drugs used for cancer chemotherapy are generally toxic to both cancer cells and normal cells and the drug treatment can cause undesired and often lethal side effects. In the treatment regimes of the present invention there was observed no evidence of normal cell death or inflammation. Further, no mice died as a result of the treatment. Most of the treated mice appeared energetic and healthy, and free of weight loss.

“Cancer” refers to all types of cancers, or neoplasms, or benign or malignant tumors. In one embodiment, those cancers that attack normal healthy blood cells or bone marrow are contemplated by the present invention. Preferred cancers for treatment using methods provided herein include carcinoma. “Carcinoma” can mean a benign or malignant epithelial tumor and includes, but is not limited to, breast carcinoma, prostate carcinoma, non-small cell lung carcinoma, colon carcinoma, CNS carcinoma, melanoma carcinoma, ovarian carcinoma, or renal carcinoma.

“Treatment of cancer” refers to application, such as administering or contacting the abovementioned “cancer” conditions with the compound combinations and methods of the present invention to cause, for example, the growth of tumors to arrest, to shrink the size of a tumor mass associated with mammalian cancers, for example, of the skin or other tissue, or to cause the cancerous tumor cells or tissue to completely remit or die and optionally fully disappear from the host mammal. A preferred host mammal is a human.

“Potentiator” or “potentiators” are materials that affect the immune system or enhance the effectiveness of compounds or methods of the present invention, see for example, U.S. Pat. No. 6,177,460. In the present invention, for example, the tumor promoting compound such as a phorbol compound can be a potentiator for any of the phenol compounds, such as 6-gingerol or 6-paradol, individually or in combination, to synergistically render them more effective as retrograde-carcinoma treatment compositions or cancer treatment compositions.

“Anti-cancer” means used against cancer or tending to arrest cancer in the treatment of cancerous cells or tissues. The compound combinations and treatment methods of the present invention provide potent anti-cancer regimens as illustrated herein.

“Retrograde-carcinoma” means moving, occurring, or performing in a backward direction or opposite to the usual direction or course of cancerous tissue. The present invention provides compositions and methods for transforming malignant cancerous cells or tissue, such as skin tumors, into a retrograde-carcinoma, that is cancerous tissue which has, for example, any or all of the following properties and characteristics: a slowed growth rate, a no growth rate, a reduced cell size or tumor size, a complete shrinkage of necrotic cells or tissue, or a sloughing-off or expulsion of the necrotic cells or tissue.

“Apoptosis”, also known as “programmed cell death,” is a series of programmed steps that cause a cell to die via “self-digestion” without rupturing and releasing intracellular contents, for example, the nucleus, chromosomes, refractile bodies, etc., into the local, that is, surrounding tissue environment. Manifestations of cell apoptosis include shrinking of the cell's cytoplasm and chromatin condensation. The compositions and treatment methods of the present invention provide an effective approach to selectively achieving apoptosis in cancerous cell lines or cancerous tissue and without harming surrounding healthy cells or healthy tissue.

“Phorbol” is 1,1a-beta, 1 b-alpha,4,4a,7a-beta,7b,8,9,9a-decahydro-4a-alpha,7b-alpha,9-beta,9a-alpha-tetrahydroxy-3-(hydroxymethyl)-1,1,6,8-alpha-tetramethyl-5H-cyclopropa(3,4)benz[1,2-e]azulen-5-one of the formula

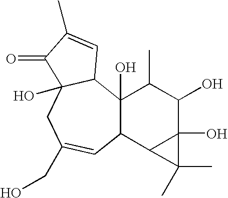

The Merck Index, 9^{th} Edition, at page 7138 under “Phorbol”, indicates that Phorbol is the parent alcohol of tumor promoting compounds in croton oil, and that phorbol diesters are potent co-carcinogens. A co-carcinogen is an agent that aggravates the carcinogenic effects of another substance.

A “gingerol compound” is any of the pungent naturally occurring compounds in the ginger plant family, or synthetic or semi-synthetic equivalents thereof, obtained for example from ginger root, such as, 6-gingerol and the like compounds illustrated herein.

A “paradol compound” is any of the pungent naturally occurring compounds in the ginger plant family, or synthetic or semi-synthetic equivalents thereof, obtained for example from ginger root, such as, 6—paradol and like compounds illustrated herein.

“Phorbol ester” is a compound or mixture of compounds with a phorbol carbocyclic ring system wherein one or more of the hydroxy groups has been acylated to form an ester, for example, the diester phorbol myristate acetate or 12-O-tetradecanoyl-phorbol-13-acetate is of the formula

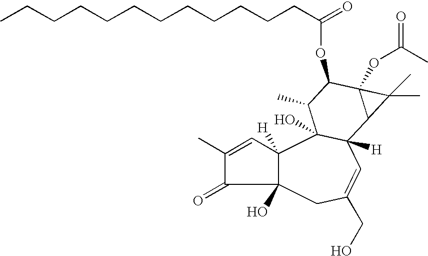

It is readily evident to one of ordinary skill in the art that the myristate and acetate ester groups or other hydroxy groups can be other known esters, for example, with from C₁-C₂₀ atoms. Phorbol esters can be prepared by acylating one or more of the hydroxy groups of phorbol with, for example, a C₁-C₂₀ saturated or unsaturated carboxylic acid or equivalent reactant. Preferred acids for forming esters, in embodiments, are, for example, tridecanoic acid or acetic acid.

The indefinite articles “a” and “an” mean “at least one” or “one or more” when used in this application including the claims unless indicated otherwise.

### DETAILED DESCRIPTION

The present invention provides in embodiments a method for the treatment of cancer, comprising:

administering to a mammal in need of such treatment a therapeutically effective amount of a combination of a compound of the formula (I)

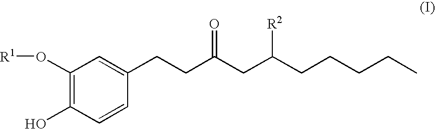

wherein R¹ is C₁-C₆ alkyl and R² is independently —H or —OH, or a pharmaceutically acceptable salt thereof; and a phorbol compound, such as a phorbol ester or diester. The phorbol ester can be, for example; 12-O-tetradecanoyl-phorbol-13-acetate. “Phorbol” and “phorbol ester” are known to those in the art with the above mentioned formulas, and can include other structurally related phorbol compounds.

In embodiments the compound of the formula (I) can be where R¹ is methyl and R² is —H, for example, of the formula (II)

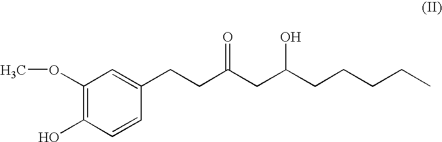

In embodiments the compound of the formula (I) can be where R¹ is methyl and R² is —H, for example, the compound of the formula (III)

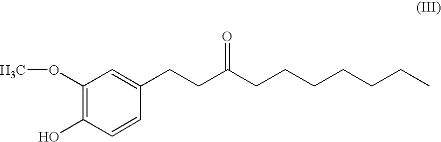

In still other embodiments the combination of the compound of formula (I) can be a mixture of two compounds where the first compound has R¹=methyl and R²=—H, and the second compound has R¹=methyl and R²=—OH, that is, a mixture of the above mentioned compounds of the formulas (II) and (III). The mixture of compounds of the formulas (II) and (III) can be administered in a weight ratio of from about 100:1 to about 1:100, and preferably from about 1:1 to about 4:1. The weight ratio of the compound or compounds of the formula (I) and the tumor promoting compound can be from about 500:1 to about 10:1, for example, when compound (I) was 6-gingerol and the tumor promoting compound was phorbol, a weight ratio of about 60:1 was effective to arrest and shrink mouse tumors.

In embodiments, the combination can be administered topically, for example, by painting or applying the combination, that is, together, severally, or sequentially, and directly onto a skin tumor mass. Other examples of topical application include applying the mixture dispersed or dissolved in a suitable carrier. Other examples of effective administration include, for example, intra-corporally, that, is within the tumor mass itself, such as by injection or implantation of a suitably adapted dosage form.

The compositions and treatment regimens of the present invention can arrest, that is partially or completely, stop or suspend, tumor growth and tumor cell proliferation, and prevent the spread of the treated tumor mass. The compositions and treatment regimens of the present invention can, for example, as a result of the above-mentioned arresting of the tumor growth and little or no toxic effects on healthy or non-diseased tissue, can cause a reduction in the size of a treated tumor. For example, the reduction in the size, or shrinkage, of a treated tumor mass can be from about 10 to about 100 percent compared to the size of the tumor before treatment.

The compositions and treatment regimens of the present invention can have, for example, very low or no apparent toxic effects on healthy cells or tissues. In embodiments, the compositions and treatment regimens of the present invention can be, for example, entirely free of toxic effects on the treated mammal, that is, there are no apparent ill-effects on surrounding healthy cells or tissue nor is there any apparent ill-effects, systemic or otherwise, on the treated mammal. Thus the present invention overcomes the above mentioned toxicity limiting aspects of other available cancer treatment methods. The compositions and treatment regimens of the present invention can used for the treatment of mammals, such as, a mouse, a dog, a farm animal, or a human.

In embodiments the present invention provides a method of inhibiting the growth of established cancer cells, such as established skin tumors, comprising: contacting the cancer cells with a cancer cell growth inhibiting amount of a mixture of compounds of the formula (I)

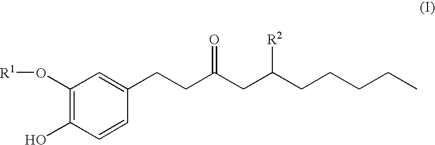

wherein R¹ is C₁-C₆ alkyl and R² is independently —H or —OH, or a pharmaceutically acceptable salts thereof; and a phorbol compound, such as phorbol or a phorbol ester.

In embodiments, the present invention also provides a method for the treatment of cancer, comprising: administering to a mammal in need of such treatment a therapeutically effective amount of a combination of 6-gingerol, 6-paradol, and 12-O-tetradecanoyl-phorbol-13-acetate. The combination can comprise from about 60 to about 90 weight percent of 6-gingerol, from about 10 to about 40 weight percent of 6-paradol, and from about 1 to about 5 weight percent of the tetradecanoyl phorbol acetate.

In embodiments the present invention also provides a method for the treatment of cancer, comprising: administering to a mammal in need of such treatment a therapeutically effective amount of a combination of 6-gingerol and 12-O-tetradecanoyl-phorbol-13-acetate. The combination can comprise from about 95 to about 99 weight percent of 6-gingerol, and from about 1 to about 5 weight percent of the phorbol acetate.

In embodiments the present invention also provides a method for the treatment of cancer, comprising: administering to a mammal in need of such treatment an effective amount of a combination of 6-paradol and 12-O-tetradecanoyl-phorbol-13-acetate. The combination can comprise from about 90 to about 99 weight percent of 6-paradol, and from about 1 to about 5 weight percent of the phorbol acetate.

In embodiments, the present invention also provides a method for the treatment of cancer, comprising: administering to a mammal in need of such treatment a therapeutically effective amount of a combination of 6-gingerol and 6-paradol. The combination can comprise from about 60 to about 90 weight percent of 6-gingerol, from about 10 to about 40 weight percent of 6-paradol. The combination can comprise, for example, a relative weight ratio of from about 60 to about 90 parts of 6-gingerol to from about 10 to about 40 parts of 6-paradol.

The foregoing treatment regimes can, in embodiments result in a substantial shrinkage of treated cancers, for example, in amounts of from about 10 to about 100 percent compared to a comparable or control untreated cancerous cell or tissue. The treatment can result in partial disappearance of the treated cancer. The treatment can, in embodiments, result in the complete disappearance of the treated cancers, such as found in chemically or radiation induced skin tumors.

The present invention can further comprise treating cancerous tumors that have been initiated in a mammal by UV radiation, such as contained in natural or synthetic light sources, a carcinogenic compound, or a combination thereof, followed by administrating one or more of the above inmentioned therapeutic combination of compounds. Thus it is evident that the compositions and treatment methods of the present invention can be used to remediate the effects of, for example, environmental or occupational causal agents of skin cancer. It is also believed that the compositions and treatment methods of the present invention can also be used to prevent, protect, or mitigate from the effects of causal agents of skin cancer. The compositions and treatment methods of the present invention can be used for their prophylactic properties in protecting or reducing the incidence of cancer in at-risk mammalian populations. Examples of UV radiation induced skin cancers can occur, for example, in humans with occupations or recreations which involve prolonged, and extensive direct or indirect exposure to sun light or to artificial light with comparable UV spectral components. Chemically induced carcinoma is also known in mammals, for example, the compound 7,12-dimethylbenz[a]anthracene of the formula

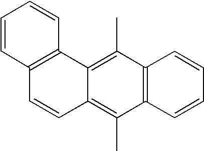

is highly effective in inducing skin tumors in mammals, such as mice. Thus the present invention provides method for the treatment of ultraviolet light or chemically induced tumors, such as skin tumors, comprising: administering to a mammal in need of such treatment an effective amount of 6-gingerol, 6-paradol, or mixtures thereof.

In embodiments, the present invention also provides a method for the treatment or prevention of UV light or chemically induced tumors, comprising: administering to a mammal in need of such treatment or prevention an effective amount of 6-gingerol or 6-paradol.

In embodiments there is provided a method of treating cancer comprising: simultaneously inhibiting activator protein (AP-1) and inducing apoptopsis in cancerous cells or tissue. The simultaneous inhibition of activator protein (AP-1) and induction of apoptopsis, in embodiments of the present invention, can be accomplished by, for example, contacting the cancer with a therapeutically effective amount of a combination of either or both 6-gingerol and 6-paradol, and a phorbol diester. Although not wanting to be limited by theory it is believed that 6-gingerol inhibits AP-1 activation and that 6-paradol induces apoptosis, while the phorbol compound, such as a phorbol diester such as TPA, in the combination with 6-paradol potentiates, that is increases the level of apoptosis. Other approaches to inhibiting AP-1 and apoptosis are known, see for example, Huang, C., Ma, W. Y., Goranson, A., and Dong, Z., in Carcinogenesis, 20: 237-42, 1999 (resveratrol suppression of cell transformation and induction of apoptosis through a p53-dependent pathway); and Dong, Z., in Biofactors, 12: 17-28, 2000 (effect of food factors on signal transduction pathways).

In embodiments the administration or contacting of the above-mentioned treatment methods can further comprise delivering the combination in any suitable carrier or diluent.

The present invention provides in embodiments a cancer treatment or prevention lotion or ointment formulation comprising: a compound of the formula (I)

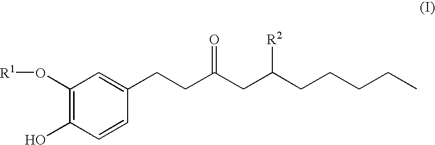

wherein R¹ is C₁-C₆ alkyl and R² is independently —H or —OH, or a pharmaceutically acceptable salt thereof; a tumor promoting phorbol compound; and a carrier or diluent. The lotion formulation containing a compound of formula (I) and a phorbol compound is a remedial formulation for use with existing or established skin tumors and in embodiments can comprise, for example, a mixture of from about 60 to about 90 weight percent of 6-gingerol and from about 10 to about 40 weight percent of 6-paradol, or pharmaceutically acceptable salts, and from about 1 to about 5 weight percent of a tumor promoting phorbol compound. Other optional ingredients include, for example, UV light absorbing compounds or blocking compounds, emollients, and the like additives, reference for example, U.S. Pat. No. 6,020,323, especially in section 4.7, and references therein. The substances of the present invention also find use in topically administered compositions, such as those preparations for the treatment of established skin cancer tumors. Beyond purely therapeutic applications, the compound combinations of the present invention may have utility in supplementing the protective action of cosmetic compositions, such as sunscreen or suntan lotions. The incorporation of the active substances of the present invention in cosmetic formulations is specifically contemplated both for the purpose of preserving and protecting the skin, as well as treating a medical condition, such as established skin tumors. In sunscreen or suntan lotion formulations it is advantageous to include an effective amount of the compound combination of the present invention along with other conventional sunscreen agents. In embodiments an effective amount of active substance is present to provide, for example, about 1 microgram to about 100 milligrams per kilogram of the mammal, preferably from about 0.01 mg to about 10 mg per kilogram of the mammal, and most preferably about 0.1 mg to about 1 mg per of the mammal. The cosmetic compositions, may contain conventional ingredients known to those of ordinary skill in the art, such as those described in Kirk-Othmer, Encyclopedia of Chemical Technology, Third Edition (1979), Vol. 7, pages 143-176. In sunscreen preparations, the addition of the compound combinations of the present invention increases the minimum erythemal dose (MED) and, consequently, the sun protection factor (SPF). Specific ingredients including typical sunscreens, are listed in, for example, the above mentioned Kirk-Othmer Encyclopedia, at pages 153-154. In addition, topical preparations and cosmetic formulations may be prepared as described in U.S. Pat. Nos. 4,199,576, 4,136,165, and 4,248,861, the disclosures of which are incorporated by reference herein in their entirety. It is apparent to those of ordinary skill in the art that the resulting compositions can be in many forms, including, but not limited to, for example, solutions, lotions, creams, pastes, emulsions, sprays, or aerosols.

The compositions and treatment methods of the present invention provide methods of treating established cancerous tumors wherein the cancer can be, for example, melanocarcinoma, colorectal cancer, hepatocarcinoma, breast cancer, prostate cancer, renal cancer, gastric cancer, esophagus cancer, lung cancer, leukemia, tongue cancer, pancreas cancer, and like cancers, and co-occurrences thereof.

The gingerol, paradol, and phorbol compounds can be, but are not limited to, those obtained by synthesis, semi-synthetic, by isolation from readily available natural product sources, or combinations thereof. Phorbol is typically used experimentally as a tumor promoter and not as a therapeutic agent. However, preliminary work in vitro unexpectedly indicated that TPA when combined with either or both 6-gingerol and 6-paradol there was induced massive tumor cell death in what appeared to be a synergistic effect. Therefore corroborative in vivo studies were accomplished where a TPA was combined with either or both 6-gingerol and 6-paradol or a pharmaceutically acceptable salt thereof, for example, subcutaneously, topically, intra-cutaneously, and the like administration routes. The results suggested that the growth of several human cancer cell lines, as measured by blocking of tumor phenotype expression, was inhibited in a concentration dependent manner when the cell lines were treated with increasing concentrations of 6-gingerol.

The invention provides a compound or compounds of the above mentioned formula (I), and a tumor promoting compound, such as phorbol and like compounds, for use in medical therapy, or alternatively, as a tool in the study and treatment of existing cancers such as established tumors in humans.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms, for example, by resolution of the racemic form by recrystallization techniques, by synthesis from optically-active starting materials, by chiral synthesis, or by chromatographic separation using a chiral stationary phase, and how to determine optical activity using the standard tests described herein, or using other similar tests which are well known in the art.

Specific and preferred values listed below for radicals, substituents, and ranges, are for illustration only; they do not exclude other defined values or other values within defined ranges for the radicals and substituents. Specifically, (C₁-C₆)alkyl can be methyl, ethyl, propyl, isopropyl, butyl, iso-butyl, sec-butyl, pentyl, 3-pentyl, or hexyl.

In the compound of the formula (I) a specific value for R¹ is methyl or —CH₃, and a specific value for R² is hydroxyl or —H, and which values taken together are 6-gingerol.

In the compound of the formula (I) a specific value for R¹ is methyl or —CH₃, and a specific value for R² is hydrogen, and which values taken together are 6-paradol.

A preferred compound of the invention is a compound of the formula (II)

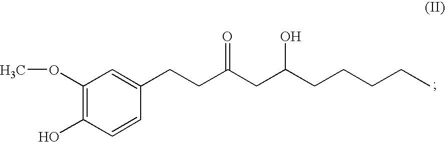

or a pharmaceutically acceptable salt thereof.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, α-glycerophosphate, and the like acids. Suitable inorganic salts may also be formed, including hydrochloride, sulfate, nitrate, bicarbonate, and carbonate salts. Pharmaceutically acceptable salts may be obtained using standard procedures known in the art, for example by reacting a sufficiently basic compound such as an amine with a suitable acid affording a physiologically acceptable anion. Alkali metal, for example, sodium, potassium or lithium, or alkaline earth metals, for example calcium salts can also be made.

The compounds of formula (I) can be formulated as pharmaceutical compositions and administered to a mammalian host, such as a human patient, in a variety of forms adapted to the chosen route of administration, that is, orally or parenterally, by intravenous, intramuscular, topical, or subcutaneous routes.

Thus, the present compounds may be systemically administered, for example, topically, in combination with a pharmaceutically acceptable vehicle such as an inert diluent or an assimilable edible carrier. The compounds may be enclosed or formulated into a variety of dosage forms such as hard or soft shell gelatin capsules, compressed into tablets, or incorporated directly with a food as part of a patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1 percent of active compound. The percentage of the compositions and preparations may, of course, be varied and may conveniently be between about 2 to about 60 percent of the weight of a given unit dosage form. The amount of active compound in such therapeutically useful compositions is such that an effective dosage level will be obtained.

The tablets, troches, pills, capsules, and the like may also contain, for example, the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose, or aspartame, or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active combination of compounds may also be administered intravenously or intraperitoneally by infusion or injection. Solutions of the active combination of compounds or their salts can be prepared in water, and optionally mixed with a nontoxic surfactant or co-solvent. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol, for example, glycerol, propylene glycol, liquid polyethylene glycols, and the like, vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable or topical compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present active combination of compounds may be applied in pure form, that is, without diluents or carrier liquids. However, it will generally be desirable to administer them to the skin as compositions or formulations, in combination with a dermatologically acceptable carrier, which may be a solid or a liquid. Useful solid carriers include finely divided solids such as talc, clay, microcrystalline cellulose, silica, alumina, and the like. Useful liquid carriers include water, alcohols or glycols or water-alcohol and glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al., U.S. Pat. No. 4,608,392; Geria, U.S. Pat. No. 4,992,478; Smith et al., U.S. Pat. No. 4,559,157; and Wortzman U.S. Pat. No. 4,820,508.

Useful dosages of the compounds of formula (I) and a phorbol compound can be determined by comparing their in vitro activity, and in vivo activity in animal models. Methods for the extrapolation of effective dosages in mice, and other animals, to humans are known to the art; see for example, U.S. Pat. No. 4,938,949. Generally, the concentration of the compound(s) of formula (I) and a phorbol compound in a liquid composition, such as a lotion, will be from about 0.1-25 weight percent, preferably from about 0.5-10 weight percent. The concentration in a semi-solid or solid composition such as a gel or a powder can be, for example, about 0.1-5 weight percent, and preferably about 0.5-2.5 weight percent. The amount of the active combination of compounds, or active salts or derivatives thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, for example, from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The compounds can be conveniently administered in unit dosage form; for example, containing 5 to 1,000 mg, conveniently 10 to 750 mg, and most conveniently, 50 to 500 mg of active ingredient per unit dosage form. In embodiments the active ingredients include either or both 6-gingerol and 6-paradol or the like compounds, in combination with a tumor promoting compound such as phorbol ester compound, and where the 6-gingerol and 6-paradol or the like compounds are present, individually or combined, in a relative amount of about 100 to about 500 fold weight percent greater than the phorbol ester compound.

Ideally, the active ingredient should be administered to achieve peak plasma concentrations of the active compound of from about 0.5 to about 75 micromolar (μM), preferably, about 1 to 50 μM, and most preferably, about 2 to about 30 μM. This may be achieved, for example, by the intravenous injection of a 0.05 to 5 percent solution of the active ingredient, optionally in saline, or orally administered as a bolus containing about 1 to about 100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, for example, into a number of discrete loosely spaced administrations; such as multiple inhalations from an insufflator or by application of a plurality of drops into the eye.

The ability of a compound, of the invention to act as anti-tumor or retrograde-carcinoma therapy may be determined using pharmacological models which are well known to the art, or using Test A described below.

Test A. Experimental results generally from Test A for representative compounds or combinations of compounds of the invention are shown generally in Table 1 and specifically in Table 2 and as described below. These results demonstrate that compound combinations of the invention have retrograde-carcinoma activity as illustrated herein. Accordingly the compound combinations of the invention are useful as therapeutic agents for the treatment of skin cancers, such as skin tumors. Additionally, compounds or combinations of the invention may be useful as pharmacological tools for the further investigation of cancer initiation, cancer promotion, and inhibition of cancer proliferation.

The compounds or compound combinations of the invention can also be administered in combination with other therapeutic agents that are effective for controlling cancerous cell or tissue growth conditions. The invention will now be illustrated by the following non-limiting Examples.

### EXAMPLE 1

Retrograde-Carcinoma Activity In Vivo Studies; Two-Stage Chemical Initiation—Promotion of Mouse Tumors

In a two-stage initiation-promotion model, live mice skin tumors were initiated by a single application of the known carcinogen, the above mentioned 7,12-dimethylbenz[a]anthracene (DMBA), followed by optional repeated topical applications of the abovementioned known tumor promoter compound TPA. Three mice groups consisted of:

Group 1—JNK1 knockout mice that were treated with DMBA followed by TPA applications and designated as (JNK1−/−)+TPA.

Group 2—JNK1 knockout mice that were treated with DMBA but not with TPA applications and designated as (JNK1−/−) No TPA.

Group 3—JNK1 wild type mice that were treated with DMBA followed by TPA applications and designated as (JNK1+/+)+TPA.

Each mouse had from 1 to 8 tumors with the largest tumor measured among all mice at 144 mm² and the smallest at 1 mm². Prior to therapeutic treatment Group 1 mice had significantly more tumors than mice in Groups 2 and 3. The tumors on the mice in Group 2 were significantly larger than those mice in Groups 1 and 3.

Therapeutic treatment included various amounts of either or both of the phenolic compounds and optionally in admixture with TPA. For example, 6-paradol in an amount of from about 25 to about 200 nanomol per mouse alone, that is, without TPA, or in admixture with TPA in an amount 5 nanograms per mouse; or 6-gingerol in an amount of from about 100 to about 350 nanomols per mouse alone, that is without TPA, or in admixture with TPA in an amount 5 nanograms per mouse; were applied by pipetting the mixture of compounds in an acetone solution onto the tumor mass. Other common modes of application can include, for example, painting on a lotion with a brush; spraying an aerosol spray or atomized liquid; taping on a bandage medicated with one or more of the active combination of ingredient compounds; subcutaneous injecting; and the like methods. In one study the phenolic compounds in combination with a TPA were applied on alternate days, that is every other day, for example: day 1—paradol and TPA; day 2—gingerol and TPA; day 3—paradol and TPA; day 4—gingerol and TPA; etc. In another study TPA was entirely excluded from the above mentioned alternate day treatment regime and instead there was administered a combination of gingerol and paradol five times per week, Monday through Friday, for 5 weeks. Tumors were measured and counted once a week and mice were photographed each week.

Table 1 summarizes the trends observed for changes in mouse tumor size in three different mouse-type groups following treatment with compounds of the formula (I) of the present invention with or without TPA present. It is evident that Group 2, with only four mice with one tumor each and a total of only 4 tumors, while Group 2 had a 100% tumor size reduction response the percentage range of size reduction was smaller, 15-65 percent, compared to Groups 1 and 3 which treatment groups included a phorbol compound and both Groups 1 and 3 provided a broader range of size reduction, 11-100 percent.

TABLE 1
Aggregate tumor size reduction in mice.

| | total | % | | % | % |
|---|---|---|---|---|---|
| | tumors | tumors | % range | tumors | tumors |
| Mouse | per | reduced | of size | with no | increase |
| Group | group | in size | reduction | change | d in size |
| Group 1 | 38 | 86.8 | 11-100 | 7.9 | 5.3 |
| JNK1 (−/−) + | | | | | |
| TPA | | | | | |
| Group 2 | 4 | 100 | 15-65 | 0 | 0 |
| JNK1 (−/−) | | | | | |
| no TPA | | | | | |
| Group 3 | 21 | 61.9 | 11-100 | 14.3 | 23.8 |
| JNK1 (+/+) + | | | | | |
| TPA | | | | | |

Table 2 provides the data of the changes in tumor size as observed in individual mice within the different mouse-type groups that is summarized in Table 1 with or without TPA treatment. A “mouse number” corresponds to an arbitrary individual mouse identification number. A “mouse number” having multiple data entries indicates that mouse had a corresponding equivalent number of tumors subjected to the indicated treatment regimen and observation. The indicated treatment regimen for each group corresponds to that indicated in Table 1 summarized above. The result illustrate that the compounds of formula (I) in combination with a tumor producing compound, such as a phorbol provide an effective tumor treatment method including arresting, shrinking, and in embodiments complete eradication of skin tumors. Time sequenced color photographs and tumor size measurement of the tumors in the treated mice over a six week period illustrated the clear effectiveness of the compound combination.

TABLE 2
Size reduction of individual mice tumors.^{1,2}

| | | | | | | | OVERALL |
|---|---|---|---|---|---|---|---|
| | Week 1 | Week 2 | Week 3 | Week 4 | Week 5 | Week 6 | % change |
| MOUSE No. | size | size | size | size | size | size | tumor size |
| Group 1 | | | | | | | |
| 13 | 24.75 | 23.1 | 22 | 20.25 | 9 | — | −63.636 |
| | 7 | 6 | 3 | 3 | 2 | — | −71.429 |
| | 4 | 3.5 | 1 | 1 | 1 | — | −75 |
| | 2.25 | 1 | 1 | 1 | 1 | — | −55.556 |
| 19 | 9 | 10 | 10 | 12 | 9 | — | 0 |
| | 9 | 4.5 | 1 | 0 | 0 | — | −100 |
| | 7.5 | 4 | 2.25 | 2.25 | 1 | — | −86.667 |
| | 1 | 2.25 | 1 | 1 | 1 | — | 0 |
| 25 | 39 | 15 | 15 | 9 | 6.25 | 4 | −89.744 |
| | 36 | 10 | 5.25 | 4 | 2.25 | 2.25 | −93.75 |
| | 3.5 | 3.5 | 3.75 | 1.44 | 1 | 0.25 | −92.857 |
| 36 | 56 | 66 | 38.5 | 25 | 24 | 20.25 | −63.839 |
| | 42 | 33 | 39 | 32.5 | 35.75 | 32.5 | −22.619 |
| | 32.5 | 12 | 10.5 | 8 | 8 | 6.25 | −80.769 |
| | 6 | 3 | 5 | 4 | 1 | 1 | −83.333 |
| | 4 | 2 | 1 | 1 | 1 | 1 | −75 |
| | 1 | 2 | 2.25 | 2.25 | 2.25 | 2.25 | 0 |
| 37 | 63 | 24.75 | 24 | 24 | 20 | 20 | −68.254 |
| | 18 | 18 | 18 | 17.5 | 17.5 | 16 | −11.111 |
| | 1 | 1 | 0 | 0 | 0 | 0 | −100 |
| 38 | 37.5 | 36 | 41.25 | 37.05 | 36 | 27 | −28 |
| | 32.5 | 30.25 | 31.5 | 31.5 | 27 | 24 | −26.154 |
| | 27 | 24 | 25 | 16 | 24 | 20 | −25.926 |
| | 18 | 9 | 7.5 | 6 | 10.5 | 10.5 | −41.667 |
| | 2.25 | 6 | 7.5 | 4 | 4 | 4 | 77.778 |
| 54 | 20 | 22 | 18 | 18 | 18 | 9 | −55 |
| | 15.75 | 12 | 13.5 | 9 | 9 | 7.5 | −52.381 |
| | 8 | 8 | 7 | 6 | 6 | 3.75 | −53.125 |
| | 2 | 2 | 1 | 1 | 1 | 1 | −50 |
| 65 | 90 | 80 | 71.25 | 40 | 35.75 | 33 | −63.333 |
| | 12.25 | 12 | 9 | 14 | 22 | 0 | −100 |
| 66 | 42 | 31.5 | 40 | 45 | 31.5 | 32.5 | −22.619 |
| | 14 | 13.5 | 12 | 12 | 12 | 12 | −14.286 |
| | 9 | 3 | 4 | 2 | 2 | 2 | −77.778 |
| | 1 | 2.25 | 2.5 | 2 | 2 | 2 | 100 |
| 69 | 16 | 9 | 9 | 7.5 | 6 | 6 | −62.5 |
| | 2 | 1 | 1 | 1 | 1 | 1 | −50 |
| | 1 | 0.25 | 0.25 | 0.25 | 0 | 0 | −100 |
| Group 2 | | | | | | | |
| 27 | 39 | 23.1 | 15.75 | 19.2 | 15.75 | 15.75 | −59.615 |
| 56 | 144 | 132.25 | 126.5 | 121 | 121 | 121 | −15.972 |
| 67 | 25 | 22.5 | 8 | 8.75 | 8.75 | 8.75 | −65 |
| 68 | 49 | 49 | 49 | 32.5 | 32.5 | 29.25 | −40.306 |
| Group 3 | | | | | | | |
| 76 | 14 | 14 | 15 | 17.5 | 17.5 | 16.5 | 0 |
| | 9 | 9 | 12.25 | 10 | 8 | 8 | −11.111 |
| | 6 | 6 | 7 | 8.75 | 7 | 2.25 | −62.5 |
| | 6 | 6 | 4 | 4 | 1 | 1 | −83.333 |
| 90 | 4 | 2.25 | 4 | 1 | 1 | — | −75 |
| 89 | 42.25 | 36 | 24 | 24 | 22 | 8.75 | −79.29 |
| | 9 | 9 | 9 | 9 | 1 | 0 | −100 |
| 83 | 48.75 | 12 | 10 | 8.75 | 6 | 6 | −87.692 |
| 100 | 9 | 6 | 3.75 | 3 | 2.25 | — | −75 |
| 108 | 9 | 9 | 9 | 18 | 18 | 16 | 77.778 |
| | 9 | 2.25 | 2.25 | 5 | 5 | 9 | 0 |
| 105 | 9 | 9 | 4 | 6 | 6 | 12.25 | 36.111 |
| | 6.25 | 1 | 0 | 0 | 0 | 0 | −100 |
| | 4 | 4 | 3 | 5 | 4 | 4 | 0 |
| 113 | 15 | 12 | 12 | 14 | 14 | 25 | 66.667 |
| 135 | 65 | 44 | 40.5 | 32.5 | 32.5 | 32.5 | −50 |
| | 16 | 9 | 7.5 | 10 | 10 | 9 | −43.75 |
| 136 | 48 | 48.75 | 35.75 | 32.5 | 48 | 65 | 35.417 |
| | 48 | 37.5 | 27 | 31 | 30.25 | 27.5 | −42.708 |
| 139 | 48 | 35 | 45 | 99 | 84 | 95 | 97.917 |
| | 48 | 33 | 27 | 42 | 42 | 0 | −100 |

¹Negative numbers indicate the percentage decrease in tumor size; “0” indicates no change; and positive numbers indicates the percentage increase in size.
²“Size” is the tumor volume in cubic millimeters (mm³) measured with a micrometer.

### EXAMPLE 2

RADIATION INITIATED MOUSE TUMORS Healthy mice were irradiated with ultraviolet (UV) light. For UV-induced skin carcinogenesis, the back of each mouse was shaved each week and then exposed to ultraviolet B radiation emitted by banks of six FS40 Westinghouse fluorescent sun lamps for 30 minute per day per 5 days per week. The dose rate of the lamp output at the distance from the backs of the mice was approximately 6.94 Joules per square meter per second (J/m²/sec) as measured with a UVX Digital radiometer with a UVX-31 sensor. The FS40 lamp emits a continuous spectrum wavelength range of about 280 to about 340 nanometers. The irradiation was continued for 30 weeks for an approximate total dose of 1.87×10 Joules per square meter (J/m²).

### EXAMPLE 3

RETROGRADE-CANCER ACTIVITY AND ABSENCE OF CYTOTOXICITY IN VIVO STUDIES The data demonstrate the effectiveness of a method of skin cancer treatment that uses topical application of a combination of 6-gingerol and 6-paradol. In these experimental mice, the combination of 6-gingerol and 6-paradol appears to be highly effective in arresting ultraviolet-induced skin tumor growth and appears to cause a marked reduction in established tumor size. Following treatment of established tumors with these compounds, the tumors became extremely dry and hard as observed in the related experiments for treating chemically induced tumors. The color of the tumors before treatment were typically pinkish-red. The color of the tumors during and after treatment changed to a white, dark gray, or black. The mice that were treated with a combination of 6-gingerol and 6-paradol compounds in accordance with the present invention showed an average decrease in tumor size of about 55 percent mass volume (cubic millimeters) after 2 weeks of treatment compared to control or untreated tumors which averaged an increase in size of about 28 percent mass volume (cubic millimeters). There was a single mouse which did not respond to the treatment regimen. The combination of compounds had no apparent toxic effects on any of the mice. Also the compounds do not appear to be toxic in cell culture as assessed by the MTT assay. The MTT assay was performed to test the cytotoxicity. The JB6 cells were maintained in exponential growth in Eagles Minimal Essential Medium (MEM) supplemented with 5% heat-inactivated fetal bovine serum (FBS). The cells were added in a 0.1 mL volume of FBS/MEM to each well of a 96 well plate and cultured for 24 hours. After 24 hour, 0.1 mL of 0.1 percent FBS/MEM containing one of several combinations of compounds was added to designated wells. The TPA was added as a 10 microliter aliquot to designated wells. Cells were incubated in the presence of these chemicals for 15 hours, 19 hours, or 36 hours. Media were removed by fast inversion of the plate and 20 microliters of 5 mg/mL MTT substrate was added into each well. Cells were incubated for an additional 4 to 6 hours and then DMSO (100 microliters per well) was added and the plate was gently rotated for 10 minutes. Absorbance at 570 nanometers was read using an Immunoreader BioTek EL311S model plate reader. The results were expressed as percentage of the control.

1) control=no cells, that is media only; for background assessment; n=6 wells

2) control cells=no chemical additives, media only; n=6 wells

3) 100 micromolar gingerol; n=4 wells

4) 200 micromolar gingerol; n=4 wells

5) 400 micromolar gingerol; n=4 wells

6) 12.5 micromolar paradol; n=4 wells

7) 25 micromolar paradol; n=4 wells

8) 50 micromolar paradol; n=4 wells

9) 100 micromolar gingerol+12.5 micromolar paradol; n=4 wells

10) 200 micromolar gingerol+25 micromolar paradol; n=4 wells

11) 400 micromolar gingerol+50 micromolar paradol; n=4 wells

12) 20 nanogram per mL TPA; n=4 wells

13) 20 nanogram per mL TPA+200 micromolar gingerol; n=4 wells

14) 20 nanogram per mL TPA+25 micromolar paradol; n=4 wells

Results from an MTT assay indicated that compounds, such as 6-gingerol or 6-paradol alone or in combinations with TPA are non-toxic, that is, they have no effect on the proliferation of cells in culture except at very high concentrations, for example, above about from about 400 to about 500 micromolar of either or both 6-gingerol or 6-paradol, and from above about 50 nanograms per milliliter of TPA.

Cell lines. The human cancer cell lines, obtained from human cancer patients, included skin cancer cells (SK-OV-3), colon adenocarcinoma (HCT-15), 2 colorectal adenocarcinoma epithelial cell lines (DLD-1 and HCT-116) and breast cancer cells (T47D).

### EXAMPLE 4

RETROGRADE CANCER IN VITRO STUDIES The results show that 6-gingerol effectively blocks tumor phenotype expression in a concentration dependent manner in a variety of human tumor cell lines. 6-Gingerol was highly effective in preventing phenotype expression of SK-OV-3 human skin cancer cells, HCT-15 human colon adenocarcinoma cells, HCT 116 human colorectal adenocarcinoma epithelial cells, and DLD-1 human colorectal adenocarcinoma epithelial cells. 6-gingerol was also effective in inhibiting phenotype expression of T47D breast cancer cells. The in vitro results demonstrate and in vivo results suggest the potential of these compound combinations, for example, for preventing or treating established tumors, for example, in humans.

### EXAMPLE 5

The following illustrate representative pharmaceutical dosage forms containing a compound or combination of compounds of the formula (I) and optionally a phorbol compound, for therapeutic use in humans. “Compound X” refers to 6-gingerol. “Compound Y” refers to 6-paradol. “Compound Z” refers to above illustrated phorbol diester.

In the exemplary formulations that follow, ‘Compounds X+Y+Z’ can be present in a formulation or dosage form, for example, in a relative weight ratio of from about 100:100:1 to about 500:500:1. ‘Compounds X+Y’ can be present in a formulation or dosage form in a relative weight ratio of from about 1:1 to about 4:1, and optionally without compound Z present. Compound X may be effective and optionally used without either compound Y or compound Z present.

| (i) Tablet 1 | mg/tablet |
|---|---|
| ‘Compounds X + Y + Z’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Compounds X + Y + Z’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compounds X + Y + Z’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| ‘Compounds X + Y + Z’ | 1.0 |
| Dibasic sodium phosphate | 12.0 |
| Monobasic sodium phosphate | 0.7 |
| Sodium chloride | 4.5 |
| 1.0 N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| ‘Compounds X + Y + Z’ | 10.0 |
| Monobasic sodium phosphate | 0.3 |
| Dibasic sodium phosphate | 1.1 |
| Polyethylene glycol 400 | 200.0 |
| 01 N Sodium hydroxide solution | |
| (pH adjustment to 7.0-7.5) | q.s. |
| Water for injection | q.s. ad 1 mL |

| (vi) Aerosol | mg/can |
|---|---|
| ‘Compounds X + Y + Z’ | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

| (vii) Lotion | mg/gram |
|---|---|
| ‘Compounds X + Y + Z’ | 20.0 |
| UV absorber or blocker | 50.0 |
| Oleic acid | 10.0 |
| Mineral oil | 1,000 |

The above formulations may be obtained by conventional preparative procedures known in the pharmaceutical art.

All publications, patents, and patent documents above are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A pharmaceutical composition comprising a combination of a compound of the formula (I) 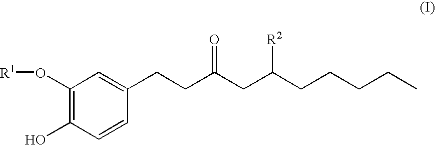
wherein R¹ is C₁-C₆ alkyl and R² is independently —H or —OH, or a pharmaceutically acceptable salt thereof; and a phorbol compound; and a pharmaceutically acceptable carrier.

2. The composition of claim 1 for use in medical treatment.

3. The composition of claim 2 wherein the treatment is the treatment of cancer or tumors.

4. The composition of claim 2 wherein the treatment is the treatment of established tumors.

5. The use of the composition of claim 1 to prepare a medicament for the treatment of cancer in a mammal.

6. The use of claim 5 wherein the treatment is the treatment of established tumors in a mammal.

7. The use of the composition of claim 1 to prepare a medicament for inhibiting established tumors in a mammal.

8. The use of claim 5 or 7 wherein the phorbol compound is 12-O-tetradecanoyl-phorbol-13-acetate.

9. The use of claim 5 or 7 wherein R¹ is methyl and R² is —OH.

10. The use of claim 5 or 7 wherein R¹ is methyl and R² is —H.

11. The use of claim 5 or 7 wherein the composition comprises a first compound of formula (I) wherein R¹ is methyl and R² is —H, and a second compound of formula (I) wherein R¹ is methyl and R² is —OH.

12. The use of claim 5 or 7 wherein the weight ratio of the compound or compounds of the formula (I) and the phorbol compound is from about 100:1 to about 500:1.

13. The use of claim 9 wherein compounds of the formula (I) are administered in a weight ratio of the first compound to the second compound from about 4:1 to about 1:1.

14. The use of claim 5 or 7 wherein the combination is administered topically.

15. The use of claim 5 or 7 wherein the treatment arrests tumor growth.

16. The use of claim 5 or 7 wherein the treatment shrinks the size of a tumor from about 10 to about 100 percent.

17. The use of claim 5 or 7 wherein the treatment is free of harmful or toxic side effects to surrounding healthy cells.

18. The use of claim 5 or 7 wherein the mammal is a mouse or a human.

19. The use of claim 5 or 7 wherein the composition comprises a mixture of 6-gingerol and 6-paradol, or pharmaceutically acceptable salts thereof; and the phorbol compound is 12-O-tetradecanoyl-phorbol-13-acetate.

20. The use of claim 5 or 7 wherein the composition comprises a relative weight ratio of the mixture of from about 60 to about 90 parts of 6-gingerol to from about 10 to about 40 parts 6-paradol, and from about 1 to about 5 parts of the 12-O-tetradecanoyl-phorbol-13-acetate.

21. A pharmaceutical composition comprising 6-gingerol and 12-O-tetradecanoyl-phorbol-13-acetate, and a pharmaceutically acceptable carrier.

22. The composition of claim 21 for use in medical treatment.

23. The composition of claim 22 wherein the treatment is the treatment of cancer or tumors.

24. The use of the composition of claim 21 to prepare a medicament for the treatment of cancer in a mammal.

25. The use of claim 24 wherein the treatment is the treatment of established tumors.

26. The use of claim 25 wherein the composition comprises from about 90 to about 99 weight percent of 6-gingerol, and from about 1 to about 5 weight percent of the 12-O-tetradecanoyl-phorbol-13-acetate.

27. A pharmaceutical composition comprising 6-paradol and 12-O-tetradecanoyl-phorbol-13-acetate, and a pharmaceutically acceptable carrier.

28. The composition of claim 27 for use in medical treatment.

29. The composition of claim 28 wherein the treatment is the treatment of cancer or tumors.

30. The use of the composition of claim 27 to prepare a medicament for the treatment of cancer in a mammal.

31. The use of claim 30 wherein the treatment is the treatment of established tumors.

32. The use of claim 31 wherein the composition comprises from about 90 to about 99 weight percent of 6-paradol, and from about 1 to about 5 weight percent of the 12-O-tetradecanoyl-phorbol-13-acetate.

33. The use of a therapeutically effective amount of a 6-gingerol, 6-paradol, or mixtures thereof to prepare a medicament for treating ultraviolet light induced tumors.

34. The use of claim 33 wherein the relative weight ratio of 6-gingerol and 6-paradol is from about 60 to about 90 parts of 6-gingerol to from about 10 to about 40 parts of 6-paradol.

35. The use of a therapeutically effective amount of a gingerol compound, a paradol compound, or combinations thereof, or a pharmaceutically acceptable salt or salts thereof, and a phorbol compound, to prepare a medicament for treating cancer by simultaneously inhibiting activator protein (AP-1) and inducing apoptopsis in cancerous cells or tissues.

36. The use according to any of claims 5 to 20, 24 to 26 and 30-35 wherein the cancer or the tumor is melanocarcinoma, colorectal cancer, hepatocarcinoma, breast cancer, prostate cancer, renal cancer, gastric cancer, esophagus cancer, lung cancer, leukemia, tongue cancer, and pancreas cancer.

37. A sunscreen formulation comprising a combination of a compound of the formula (I) 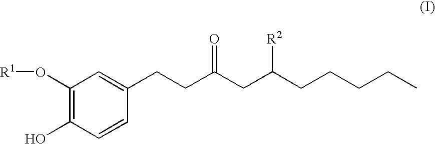
wherein R¹ is C₁-C₆ alkyl and R² is independently —H or —OH, or an acceptable salt thereof; and a phorbol compound; and an acceptable carrier.

38. A cosmetic formulation comprising a combination of a compound of the formula (I) 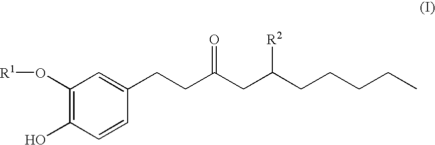
wherein R¹ is C₁-C₆ alkyl and R² is independently —H or —OH, or an acceptable salt thereof; and a phorbol compound; and an acceptable carrier.

39. The formulation of claims 37 or 38 wherein the compound of the formula (1) is a mixture of from about 60 to about 90 weight percent of 6-gingerol and from about 10 to about 40 weight percent of 6-paradol, or acceptable salts thereof, and from about 1 to about 5 weight percent of a tumor promoting phorbol compound.
